(19) United States
(12) Patent Application Publication
(10) Pub. No.: US 2019/0099616 A1
(43) Pub. Date: Apr. 4, 2019

(54) OMNIBLANKET FOR INFANT WARMING AND INCREASED EFFICIENCY PHOTOTHERAPY

(71) Applicant: GENERAL ELECTRIC COMPANY, Schenectady, NY (US)

(72) Inventors: Nagapriya Kavoori Sethumadhavan, Bangalore (IN); Steven Falk, Baltimore, MD (US)

(21) Appl. No.: 15/723,333

(22) Filed: Oct. 3, 2017

(51) Int. Cl. A61N 5/06; A61F 7/00

(52) U.S. Cl. CPC A61N 5/0621; A61N 2005/0659; A61N 2005/0662; A61F 7/0097

(57) ABSTRACT

A blanket for use in phototherapy treatment integrates a phototherapy element for delivering phototherapy to a patient placed on the blanket and a heating element for warming the patient. In one example, the phototherapy element includes a light emitting panel made from woven optical fibers and the heating element includes a heating wire secured to the light emitting panel. In another example, both phototherapy and warming are provided by a woven optical fiber panel. Blue light is emitted from the panel for phototherapy and infrared light is emitted from the panel for warming.

### BACKGROUND

This disclosure relates to devices and methods used in the treatment of neonatal hyperbilirubinemia (infant jaundice).

Livers of some newborns are not mature enough to filter out bilirubin. Excessive bilirubin accumulated in the blood results in hyperbilirubinemia (jaundice), which may cause brain damage and even death. Phototherapy is an effective method for treating neonatal hyperbilirubinemia where bilirubin molecules absorb light in the blue spectra (e.g., wavelength of 425-475 nm) and convert into water soluble isomers which are then excreted by the body. For phototherapy treatment to be effective, the blue light needs to penetrate the skin to reach bilirubin molecules in the blood. However, a portion of the blue light is absorbed by the skin, resulting in reduced phototherapy efficiency. Improvement of phototherapy efficiency is generally desired.

### SUMMARY

In one embodiment, the present disclosure provides a blanket for use in phototherapy treatment. The blanket comprises a phototherapy element configured to deliver phototherapy to a patient placed on the blanket and a heating element configured to warm the patient based on a temperature of the patient.

In another embodiment, the present disclosure provides a device for phototherapy treatment. The device comprises a blanket, a controller, and a power supply. The blanket comprises a phototherapy element configured to deliver phototherapy to a patient placed on the blanket and a heating element configured to warm the patient. The controller comprises a phototherapy controller configured to control operation of the phototherapy element and a heating controller configured to control operation of the heating element. The power supply is configured to supply power to the phototherapy element and the heating element.

In yet another embodiment, the present disclosure provides a method for providing phototherapy treatment. The method comprises providing a blanket, the blanket comprising a phototherapy element configured to deliver phototherapy and a heating element configured to warm the patient. The method also comprises delivering phototherapy to a patient placed on the blanket with the phototherapy element and warming the patient with the heating element.

### BRIEF DESCRIPTION OF THE DRAWINGS

Various aspects of this disclosure may be better understood upon reading the following detailed description and upon reference to the drawings in which:

FIG. 1 is a block diagram of a phototherapy device, in accordance with an exemplary embodiment;

FIG. 2A is a schematic plan view of a blanket that can be used in the phototherapy device of FIG. 1, in accordance with an exemplary embodiment;

FIG. 2B is a schematic cross-sectional view of the blanket of FIG. 2A along line A-A′, in accordance with an exemplary embodiment;

FIG. 2C is a schematic diagram of a heating element that can be used in the blanket of FIG. 2A, in accordance with an exemplary embodiment;

FIG. 3A is a schematic plan view of a blanket that can be used in the phototherapy device of FIG. 1, in accordance with another exemplary embodiment;

FIG. 3B is a schematic cross-sectional view of the blanket of FIG. 3A along line B-B′, in accordance with an exemplary embodiment;

FIG. 4 is a block diagram of a controller that can be used in the phototherapy device of FIG. 1, in accordance with an exemplary embodiment;

FIG. 5 is a block diagram of a controller that can be used in the phototherapy device of FIG. 1, in accordance with another exemplary embodiment;

FIG. 6 is a flow chart of a method for providing phototherapy treatment by using the phototherapy device of FIG. 1, in accordance with an exemplary embodiment.

The drawings illustrate specific aspects of the described components, systems and methods for providing phototherapy treatment. Together with the following description, the drawings demonstrate and explain the principles of the structures, methods, and principles described herein. In the drawings, the thickness and size of components may be exaggerated or otherwise modified for clarity. Well-known structures, materials, or operations are not shown or described in detail to avoid obscuring aspects of the described components, systems and methods.

### DETAILED DESCRIPTION

One or more specific embodiments of the present disclosure are described below in order to provide a thorough understanding. These described embodiments are only examples of the systems and methods for providing phototherapy treatment. The skilled artisan will understand that specific details described in the embodiments can be modified when being placed into practice without deviating the spirit of the present disclosure.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

Referring to the figures generally, the present disclosure is to provide devices and methods for phototherapy treatment with improved efficiency. An exemplary device includes a blanket that includes a phototherapy element for delivering phototherapy to an infant placed on, or at least partially surrounded by, the blanket and a heating element for warming the infant. Blue light, when penetrating the skin to reach bilirubin molecules in the blood for phototherapy, is partially absorbed by the skin. Heat can dilate blood vessels (i.e., vasodilation), which can increase absorption of the blue light by the blood because the volume of blood near the skin surface turns higher. Thus, simultaneously warming the infant and delivering phototherapy can improve the phototherapy efficiency. Additionally, a preterm or low birth weight neonate (or a neonate with complications) generally needs interventions such as phototherapy and extra warming for thermoregulation.

In addition, a blanket integrating the phototherapy element and warmer enables developmental care of the infant. Kangaroo care, involving skin to skin contact between the infant and the mother, is an important component of developmental care. The blanket as disclosed herein is easily portable—the infant may be carried within the blanket. The infant may be comforted and/or transported by a caregiver while the phototherapy element and warmer are in use. Therefore, the developmental/Kangaroo care can be delivered without stopping the phototherapy and warming.

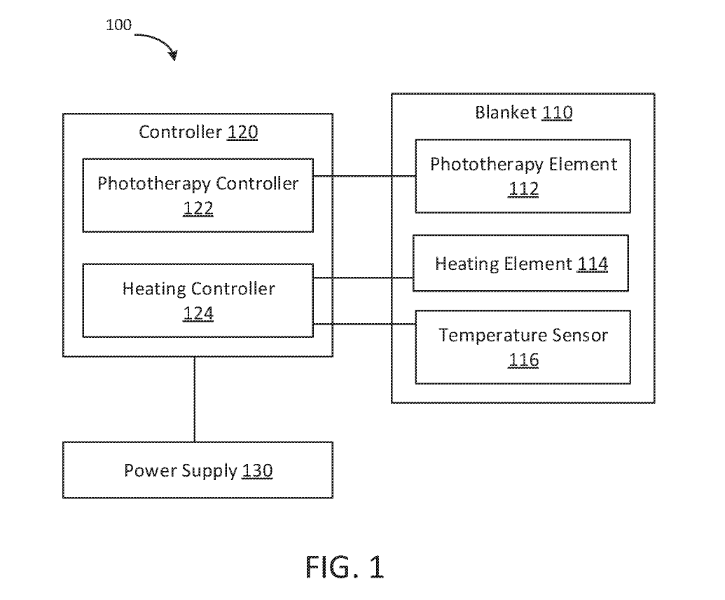

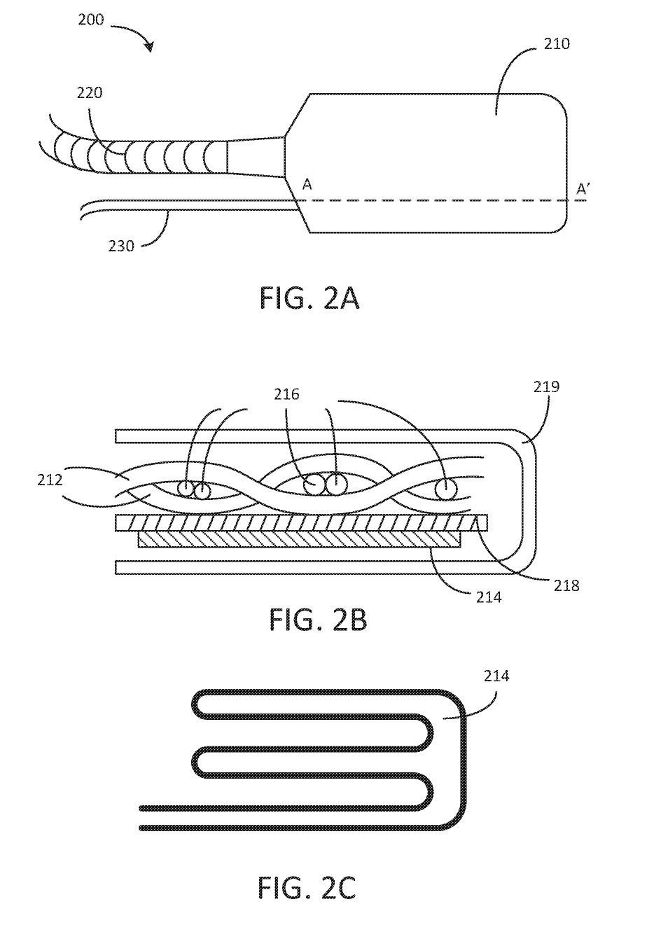

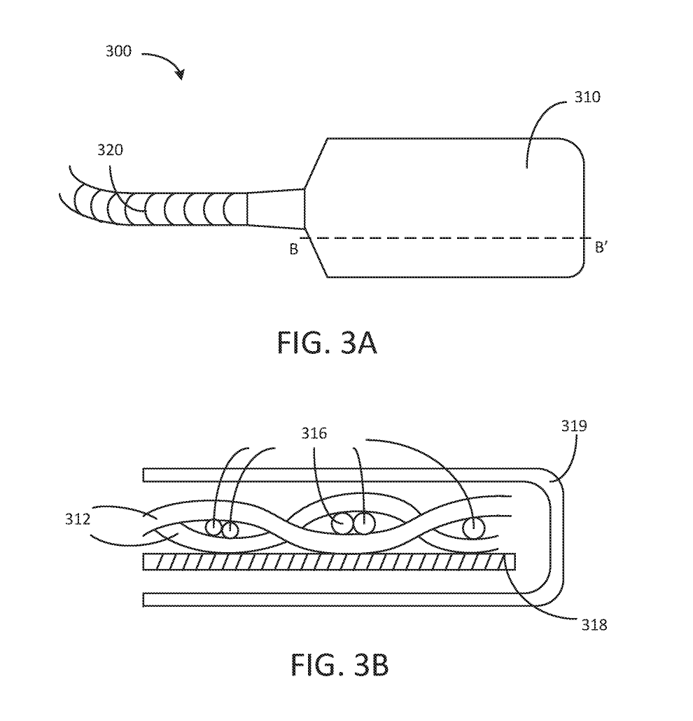

Now referring to FIG. 1, a block diagram of a phototherapy device 100 is shown, in accordance with an exemplary embodiment. As illustrated in FIG. 1, in some embodiments, the phototherapy device 100 includes a blanket 110, a controller 120, and a power supply 130. The blanket 110 can be used in close proximity to an infant to deliver phototherapy with a phototherapy element 112, and warm the infant with a heating element 114. The blanket 110 may be sized in an exemplary embodiment such that it can be wrapped around the infant to at least partially surround the infant. The infant can also be carried in the blanket 110 for Kangaroo care without stopping phototherapy and warming. The blanket 110 further includes a temperature sensor 116, e.g., a thermocouple. In operation, the temperature sensor 116 may contact the skin of the infant to monitor the temperature of infant and provide feedback to the controller 120. Structure of the blanket 110 is discussed in further detail below with reference to FIGS. 2A-2C and FIGS. 3A-3B.

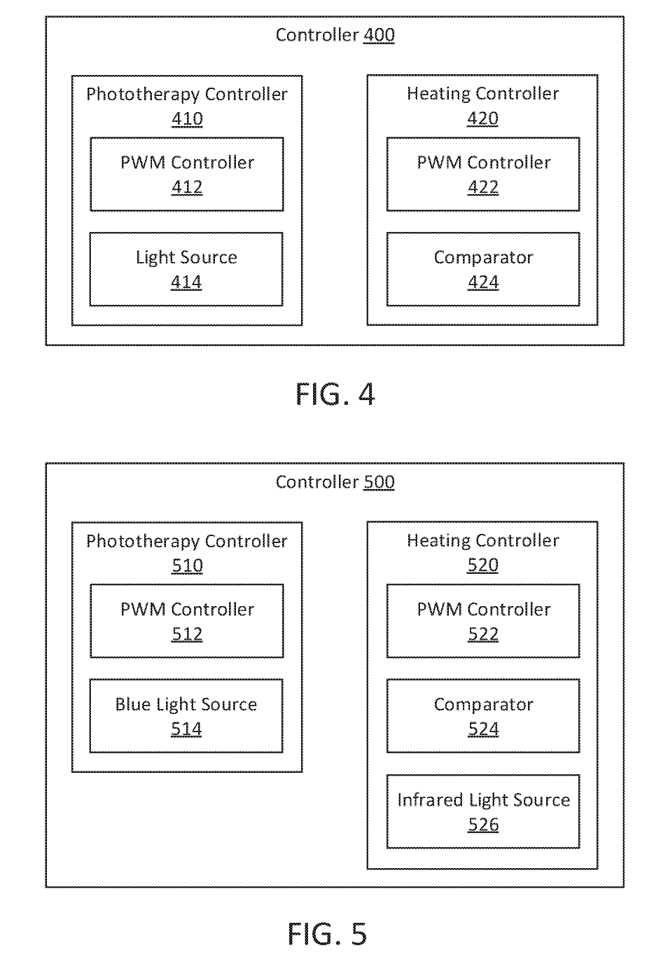

The controller 120 includes a phototherapy controller 122 configured to control the operation of the phototherapy element 112 and a heating controller 124 configured to control the operation of the heating element 114 based on the infant's temperature monitored by the temperature sensor 116. Structure of the controller 120 is discussed in further detail below with reference to FIGS. 4-5.

The power supply 130 is configured to supply power to the phototherapy element 112 through the phototherapy controller 122 and to the heating element 114 through the heating controller 124. In some embodiments, the power supply 130 includes a direct current (DC) power supply, such as a battery pack, so that the phototherapy device 100 can be highly portable. In some embodiments, the power supply 130 includes an alternating current (AC) power supply, such as an AC-DC adapter that can be plugged into an AC wall outlet. In some embodiments, the power supply 130 includes both DC power supply and AC power supply. In such embodiment, when the infant is in bed, the AC power supply is plugged into the AC wall outlet and the DC power supply (e.g., rechargeable battery) can be charged and when the infant is moved around, the AC power supply is unplugged and the DC power supply is put in use. In some embodiments, the controller 120 and the power supply 130 are enclosed in a housing (e.g., a box) outside of the blanket 110.

Referring to FIGS. 2A-2C, a schematic plan view of a blanket 200 is shown in FIG. 2A, in accordance with an exemplary embodiment. The blanket 200 can be used as the blanket 110 in the phototherapy device of FIG. 1. FIG. 2B shows a schematic cross-sectional view of the blanket 200 along line A-A′. FIG. 2C shows a schematic diagram of a heating element used in the blanket 200.

As illustrated in FIGS. 2A-2C, in some embodiments, the blanket 200 includes a light emitting panel 210 made from one or more layers of optical fibers 212 woven into a sheet or mat. In use, an infant is placed on the light emitting panel 210 to receive phototherapy. The optical fibers at one end of the panel 210 are brought together and bundled to form a cable (e.g., ribbon cable or round cable) 220. The cable 220 can transmit light from a remote light source (e.g., located in the housing with the phototherapy controller) to the panel 210 for providing phototherapy to the infant. In some embodiments, the heating element 214 includes a heating wire made of a resistive conductor (e.g., copper nickel alloy) and is secured to the back surface of the panel 210 through, for example, adhesive. In some embodiments, the adhesive is highly heat conductive to further facilitate conduction of the heat generated by the heating element 214. The heating element 214 is connected to the heating controller through a cable 230. It should be understood the heating element 214 can be of different shapes from that shown in FIG. 2C. The woven fiber panel 210 and the heating wire 214 are flexible so that the blanket 200 can be wrapped as desired.

Each of the optical fibers 212 may be made from one or more optical fiber strands, each strand including a light transmitting core portion of a first transparent material and an outer sheath or cladding of a second transparent material. The sheath material (i.e., the second transparent material) has a lower index of refraction than that of the core material (i.e., the first transparent material) in order to prevent the escape of light. The core material can be made of either glass or plastic or a multi-strand filament having the desired optical characteristics. Because the index of refraction of the sheath material is lower than that of the core material, substantially total reflection is achieved at the sheath-core interface. To cause light to be emitted from the light emitting panel 210, the optical fibers 212 are bended at a plurality of locations along the length. The angle of each bend of the optical fibers 212 approximately exceeds the angle of internal reflection so that a portion of the light can be emitted at each bend. In some embodiments, the optical fibers 212 are woven in the warp direction, with fill threads 216 woven in the weft direction and crossed by the optical fibers 212 to form the bends at the optical fibers 212. The fill threads 216 can be made of conventional fibers, such as cotton, nylon, wool, and the like. In some embodiments, the fill threads 216 are made of a transparent thermoplastic so that they do not interfere with the light transmitted towards the infant. In some embodiments, the fill threads 216 are made of thermally conductive polymers or metals or alloys to further facilitate conduction of the heat generated by the heating element 214. In some other embodiments, the fill threads 216 can be made of transparent and thermally conductive materials so that they do not interfere with the light or heat transmitted towards the infant.

The light output pattern from the panel 210 can be varied by changing the weave spacing and pattern of the woven optical fibers 212 as well as the shape and radius of the bends at various locations. For example, the illumination can be increased by placing the bends closer together or by making the weave progressively tighter as the distance from the cable 220 increases. Fill threads 216 with different coefficients of friction can be used to help control the tightness of the weave, in that the higher the coefficient of friction, the tighter it is possible to weave the optical fibers 212. In addition, more than one fill threads 216 may be used at the bend to provide more surface points for increased friction, and to reduce the thickness of each individual fill thread 216 and thus the thickness of the panel 210 while achieving substantially the same rigidity provided by a thicker fill thread. In some embodiments, a reflective layer 218 is disposed adjacent to the panel 210 to direct the scattered light toward the infant.

In some embodiments, the optical fibers 212 are coated with a material (not shown in the present Figures) that can change the attenuation of the optical fibers 212. The amount of attenuation can be varied by varying the index of refraction and thickness of the applied coating. In some embodiments, the coating is applied to the entire length of the optical fibers 212 so that attenuation changes occur over the entire light emitting portion. In other embodiments, only selected areas of the bends of the optical fibers 212 are coated with the coating to change the attenuation of the light emitted from the selected areas.

In some embodiments, the light emitting panel 210 is at least partly surrounded with a cover 219 as a contamination barrier between the panel 210 and the skin of the infant. In some embodiments, the cover 219 includes a disposable overwrap made from thin biocompatible polymer, such as polyethylene, polyurethane or cellophane, and is transparent so as not to substantially reduce the intensity of light transmitted to the infant. In some embodiments, the cover 219 is made from thin washable fabric, such as cotton, nylon, or the like. The cover 219 can be loosely fitted over the panel 210 in any form and can be secured by tape, elastic, or other means, and thus easily removed and disposed of or laundered for sanitary purposes.

As discussed above, the heating element 114 includes heating wires made of a resistive conductor such as copper nickel alloy, which provides a distributed heating along the length of the wire when electrical power is applied thereto. An insulation layer (not shown in the present Figure) surrounds the heating wire, which is electrically nonconductive but thermally conductive.

Referring to FIGS. 3A-3B, a schematic plan view of a blanket 300 is shown in FIG. 3A, in accordance with another exemplary embodiment. The blanket 300 can be used as the blanket 110 in the phototherapy device of FIG. 1. FIG. 3B shows a schematic cross-sectional view of the blanket 300 along line B-B′.

Different from the blanket 200 as illustrated in FIGS. 2A-2C, in the blanket 300, no heating wire is used as the heating element. Rather, both phototherapy and warming are provided through a light emitting panel 310 made from woven optical fibers 312. In particular, a cable 320 transmits blue light (e.g., wavelength of 425-475 nm) from a first remote light source and infrared light (e.g., wavelength of −1 μm or longer) from a second remote light source to the panel 310. The blue light provides phototherapy to the infant placed on the blanket 300 while the infrared light generates heat to warm the infant.

Like optical fibers 212 of the panel 210, optical fibers 312 of the panel 310 may be made from one or more optical fiber strands, each strand including a light transmitting core portion of a first transparent material and an outer sheath or cladding of a second transparent material. To cause light (i.e., both blue light and infrared light) to be emitted from the panel 310, the optical fibers 312 are bended at a plurality of locations along the length. The angle of each bend of the optical fibers 312 approximately exceeds the angle of internal reflection so that a portion of the light can be emitted at each bend. The optical fibers 312 are crossed by fill threads 316 to form the bends at various locations. In some embodiments, each of the optical fibers 312 is used to transmit both the blue light for phototherapy and the infrared light for warming. In other embodiments, a first portion of the optical fibers 312 are used to transmit the blue light while a second portion of the optical fibers 312 are used to transmit the infrared light. In further embodiments, the angle and/or shape of the bends of the first portion of the optical fibers 312 are different from those of the second portion of the optical fibers 312. In some embodiments, two types of fibers are used—a first type of fibers are used to transmit blue light while a second type are used to transmit the infrared light. The materials of the first type and the second type of fibers may be different to efficiently transmit the desired wavelengths of light. The two types of fibers can be interwoven with the desired angle and/or shape of bends to provide optimum transmission of blue light and infrared light to the infant.

In some embodiments, a reflective layer 318 is disposed adjacent to the panel 310 to direct the scattered light toward the infant. In some embodiments, the optical fibers 312 are coated with a material (not shown in the present Figures) that can change the attenuation of the optical fibers 312. The coating may be applied to the entire length of the optical fibers 312 or selected areas of the bends of the optical fibers 312. In some material, the panel 310 is at least partly surrounded with a cover 319 as a contamination barrier between the pad 210 and the skin of the infant. The cover 219 may be a disposable overwrap made from thin biocompatible polymer or a washable cover made from thin fabric.

Referring to FIG. 4, a block diagram of a controller 400 is shown in accordance with an exemplary embodiment. The controller 400 can be used as the controller 120 in the phototherapy device 100 of FIG. 1 when the blanket 200 of FIGS. 2A-2C is used. As illustrated in FIG. 4, in some embodiments, the controller 400 comprises a phototherapy controller 410 which includes a pulse width modulation (PWM) controller 412 (first PWM controller) and a light source 414, and a heating controller 420 which includes a PWM controller 422 (second PWM controller) and a comparator 424.

In operation, the PWM controller 412 controls the intensity of the phototherapy light from the light source 414 by changing the duty cycle of the power (e.g., from the power supply) provided to the light source 414. The greater the duty cycle, the higher the intensity of the phototherapy light. In some embodiments, the phototherapy controller 400 is an open-loop controller, that is, a user (e.g., a physician) sets a desired light intensity for the infant. For example, the user measures the bilirubin level of the infant twice a day and adjusts the desired phototherapy light intensity based on the measurements. In order to obtain the accurate bilirubin level, the user can measure the level after the phototherapy and heating are off for at least ten minutes. In some embodiments, the light source 414 includes a light-emitting diode (LED), which is configured to emit high-intensity blue light suitable for treating neonatal hyperbilirubinemia. It should be understood that light source other than LED can also be used.

The heating controller 420 may be closed-loop, open-loop, or configured for a combination thereof. In some embodiments, where the heating controller 420 is a closed-loop controller, the PWM controller 420 controls the heat generated to warm the infant by changing the duty cycle of the power (e.g., from the power supply) provided to the heating element (e.g., the heating element 214) based on an output from the comparator 424. The comparator 424 compares the real-time temperature of the infant monitored by a temperature sensor (e.g., temperature sensor 116) with a predefined reference temperature. If the real-time temperature is higher than the reference, the PWM controller 422 decreases the duty cycle to reduce the heat generated. If the real-time temperature is lower than the reference, the PWM controller 424 increases the duty cycle to increase the heat generated.

Referring to FIG. 5, a block diagram of a controller 500 is shown in accordance with an exemplary embodiment. The controller 500 can be used as the controller 120 in the phototherapy device 100 of FIG. 1 when the blanket 300 of FIGS. 3A-3B is used. As illustrated in FIG. 5, in some embodiments, the controller 500 comprises a phototherapy controller 510 which includes a pulse width modulation (PWM) controller 512 (first PWM controller) and a blue light source (first light source) 514, and a heating controller 520 which includes a PWM controller 522 (second PWM controller), a comparator 524, and an infrared light source (second light source) 526.

For the phototherapy controller 510, the PWM controller 512 and blue light source 514 can operate in the similar way to the PWM controller 412 and light source 414, respectively. For the heating controller 520, the PWM controller 520 controls the heat generated to warm the infant by changing the duty cycle of the power (e.g., from the power supply) provided to the infrared light source 526 based on an output from the comparator 524. The comparator 524 compares the real-time temperature of the infant monitored by a temperature sensor (e.g., temperature sensor 116) with a predefined reference temperature. If the real-time temperature is higher than the reference, the PWM controller 522 decreases the duty cycle to reduce the light intensity (and thus the generated heat) from the infrared light source 526. If the real-time temperature is lower than the reference, the PWM controller 524 increases the duty cycle to increase the light intensity (and thus the generated heat) from the infrared light source 526.

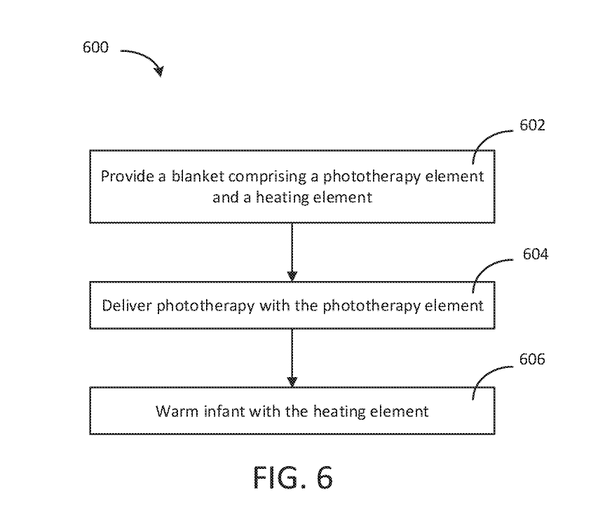

Referring to FIG. 6, a flow chart 600 of a method for providing phototherapy treatment is shown in accordance with an exemplary embodiment. At an operation 602, a blanket comprising a phototherapy element and a heating element is provided. In some embodiments, the blanket has the same structure as the blanket 200 shown in FIGS. 2A-2C. In particular, the blanket includes a light emitting panel formed by woven optical fibers and a heating wire secured to the panel. The optical fibers can transmit light for phototherapy and the heating wire can generate heat. In other embodiments, the blanket has the same structure as the blanket 300 shown in FIGS. 3A-3B. In particular, the blanket includes a light emitting panel formed by woven optical fibers for providing both phototherapy and warming. At least some of the optical fibers are used to transmit blue light for phototherapy and at least some of the optical fibers are used to transmit infrared light for warming. In some embodiments, at least some optical fibers are used to transmit both phototherapy light and the heating light. In other embodiments, a first portion of the optical fibers are used to transmit the phototherapy light while a second portion of the optical fibers are used to transmit the heating light.

At an operation 604, phototherapy is delivered to an infant with the phototherapy element. In some embodiments, an open-loop control scheme is used to control the phototherapy. For example, the user measures the bilirubin level of the infant twice a day and sets the desired phototherapy light intensity based on the measurements. The phototherapy light intensity can be changed by changing the duty cycle of the power provided to the phototherapy element. The greater the duty cycle, the higher the intensity of the phototherapy light.

At an operation 606, the infant is warmed with the heating element. In some embodiments, a closed-loop control scheme is used to control the heat generated. For example, the temperature of the infant is monitored at real time and compared to a predefined reference temperature. If the real-time temperature is higher than the reference, the heat generated is reduced. If the real-time temperature is lower than the reference, heat generated is increased. The generated heat can be changed by changing the duty cycle of the power provided to the heating element.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

What is claimed is:

1. A blanket for use in phototherapy treatment, the blanket comprising:
a phototherapy element configured to deliver phototherapy to a patient placed on the blanket; and
a heating element configured to warm the patient based on a temperature of the patient.

2. The blanket of claim 1, further comprising a temperature sensor configured to monitor the temperature of the patient.

3. The blanket of claim 1, wherein the phototherapy element includes a light emitting panel made from woven optical fibers, the heating element includes a heating wire secured to the light emitting panel, and the blanket is flexible.

4. The blanket of claim 3, wherein the light emitting panel further includes fill threads crossed by the optical fibers to form bends at a plurality of locations that emit light for phototherapy.

5. The blanket of claim 3, further comprising a reflective layer adjacent to the light emitting panel and configured to direct the light emitted from the light emitting panel to the patient placed on the blanket.

6. The blanket of claim 3, further comprising a cover at least partly surrounding the light emitting panel.

7. The blanket of claim 1, comprising a light emitting panel made from woven optical fibers, wherein the phototherapy element includes at least some of the optical fibers from which a first light for phototherapy treatment is emitted, the heating element includes at least some of the optical fibers from which a second light for warming is emitted, and the blanket is flexible.

8. The blanket of claim 7, wherein the first light includes blue light and the second light includes infrared light.

9. The blanket of claim 7, wherein at least some of the optical fibers are used for both phototherapy treatment and warming.

10. The blanket of claim 7, wherein a first portion of the optical fibers are used for phototherapy treatment and a second portion of the optical fibers are used for warming, a shape of bends on the first portion of the optical fibers is different from a shape of bends on the second portion of the optical fibers, and the first portion of optical fibers are interwoven with the second optical fibers.

11. The blanket of claim 7, wherein a first portion of the optical fibers are used for phototherapy treatment and a second portion of the optical fibers are used for warming, a material of the first portion of the optical fibers is different from a material of the second portion of the optical fibers, and the first portion of optical fibers are interwoven with the second portion of optical fibers.

12. A device for phototherapy treatment, the device comprising:
a blanket comprising:
a phototherapy element configured to deliver phototherapy to a patient placed on the blanket; and
a heating element configured to warm the patient;
a controller comprising:
a phototherapy controller configured to control operation of the phototherapy element; and
a heating controller configured to control operation of the heating element; and
a power supply configured to supply power to the phototherapy element and the heating element.

13. The device of claim 12, further comprising a temperature sensor configured to monitor a temperature of the patient, wherein the heating controller is configured to control operation of the heating element based on the temperature of the patient.

14. The device of claim 12, wherein the phototherapy element includes a light emitting panel made from woven optical fibers, and the heating element includes a heating wire secured to the light emitting panel.

15. The device of claim 12, wherein the blanket comprises a light emitting panel made from woven optical fibers, the phototherapy element includes at least some of the optical fibers from which a first light for phototherapy treatment is emitted, and the heating element includes at least some of the optical fibers from which a second light for warming is emitted.

16. The device of claim 12, wherein the phototherapy controller is an open-loop controller, and the heating controller is a closed-loop controller.

17. A method for providing phototherapy treatment, the method comprising:
providing a blanket, the blanket comprising:
a phototherapy element configured to deliver phototherapy; and
a heating element configured to warm the patient;
delivering phototherapy to a patient placed on the blanket with the phototherapy element; and
warming the patient with the heating element.

18. The method of claim 17, wherein the phototherapy element includes a light emitting panel made from woven optical fibers, and the heating element includes a heating wire secured to the light emitting panel.

19. The method of claim 17, wherein the blanket comprises a light emitting panel made from woven optical fibers, the phototherapy element includes at least some of the optical fibers from which a first light for phototherapy treatment is emitted, and the heating element includes at least some of the optical fibers from which a second light for warming is emitted.

20. The method of claim 17, wherein delivering phototherapy treatment includes using an open-loop control scheme to control a light intensity for the phototherapy treatment, and warming the patient includes using a closed-loop control scheme to control heat generated, and the method further comprises using pulse width modulation (PWM) to control at least one of the light intensity for the phototherapy treatment and the heat generated to warm the patient.
